(19) United States
(12) Patent Application Publication
(10) Pub. No.: US 2003/0143289 A1
(43) Pub. Date: Jul. 31, 2003

(54) Herbal composition PHY828 and its use

(75) Inventor: Shaohong Chen, Chengdu City (CN)

Correspondence Address: MORGAN LEWIS & BOCKIUS LLP, 1111 PENNSYLVANIA AVENUE NW, WASHINGTON, DC, 20004, US

(73) Assignee: The Affiliated Hospital of Chengdu University of Traditional Chinese Medicine

(21) Appl. No.: 10/299,009

(22) Filed: Nov. 19, 2002

Related U.S. Application Data
(63) Continuation-in-part of application No. PCT/CN01/00739, filed on May 14, 2001.

(51) Int. Cl. A61K 35/78

(52) U.S. Cl. 424/728; 424/779

(57) ABSTRACT

The present invention relates to herbal compositions and herbal extracts useful for treating cerebral vascular disease, including stroke, intracranial hemorrhage, intracranial infarction, and vascular dementia. The present invention can be used to reduce mortality rate and improve the quality of life of an individual after pathological injury that results in cerebrovascular and/or neuronal disorders. Further, the invention relates to the treatment of acute intracerebral hemorrhage by the novel herbal formulation PHY828 and provides methods of making PHY828 compositions.

### RELATED APPLICATIONS

This application is a Continuation-in-Part of International Application PCT/CN01/00739, filed May 14, 2001, which designates the U.S. of America and which is incorporated by reference in its entirety.

This application is related to Republic of China Patent Application No.90113816, filed Jun. 7, 2001, which is incorporated by reference in its entirety.

### FIELD OF THE INVENTION

The present invention relates to herbal compositions and herbal extracts useful for treating cerebrovascular disease, including head injuries, stroke, intracranial hemorrhage, intracranial infarction, and vascular dementia. The present invention can be used to reduce mortality rate and improve the quality of life of an individual after pathological injury that results in cerebrovascular and/or neural disorders.

### BACKGROUND OF THE INVENTION

All publications and patent applications herein are incorporated by reference to the same extent as if each individual publication or patent application was specifically and individually indicated to be incorporated by reference.

Herbal Medicine

Herbal medicine has been in use for centuries by people of Asia and Europe. In the United States (US), herbs have become commercially valuable in the dietary supplement industry as well as in holistic medicine. Approximately one third of the US population has tried some form of alternative medicine at least once (Eisenberg et al., N. Engl. J. Med (1993) 328: 246-252; Eiserberg et al., JAMA, (1998) 280(18): 1569-1579). Botanicals have also become a focal point for the identification of new active agents to treat diseases. Active compounds, derived from plant extracts, are of continuing interest to the pharmaceutical industry. For example, taxol an antineoplastic drug obtained from the bark of the western yew tree, has been found to be useful in the treatment of breast cancer (Gomez-Espuch et al., Bone Marrow Transplant, (2000) 25(3):231-235).

There are many branches of herbal medicine around the world, such as Ayurveda, Unani, Sida and Traditional Chinese medicine (TCM). While modem western medicine typically consists of administering a single chemical entity capable of intervening a specific biochemical pathway, each formula of TCM contains hundreds of chemical entities from several herbs which are designed to interact with multiple targets in the body in a coordinated manner. Although empirical practice contributed in a significant way to herbal composition and prescription of these ancient herbal medicines, they are also supported, to a varying degree, by a set of theories which all are distinct from that of modem western medicine in terms of anatomy, pharmacology, pathology, diagnosis treatment, etc. Among the different herbal medicine fields, TCM has developed a more complete set of theories over 2000 years of application. These theories are well documented and practiced by local physicians caring for a huge population (>1.3 billion people) in China and in East Asia including Korea, Japan. (V. R. Pelletier, The Best Alternative Medicine, 2000, Simon & Schuster).

Traditional Chinese Medicine

Western medicine generally uses purified compounds, either natural or single synthetic, mostly directed towards a single physiological target. However, the compositions used in TCM are usually composed of multiple herbs and compounds which are aimed at multiple targets in the body based on unique and holistic concepts. TCM mainly used processed crude natural products, with various combinations and formulations, to treat different conformations resulting in fewer side effects. The great potential of TCM has yet to be realized for the majority of the world's people.

The herbs in a typical TCM prescription are assigned roles as the principal herb and the secondary herbs, including assistant, adjuvant and guiding herbs. Some formulas contain up to 20 herbs or more; however, each herb in a formula can be assigned to one of the described roles (H. Y. Hsu and C. S. Hsu, Commonly used Chinese Herbal Formulas with Illustrations, Part I, Oriental Healing Arts Institute, Los Angeles, 1980). Further, according to folklore, for over two thousand years there have been about 500 herbs in use. Each of the 300 herbs can be selected to play a role in an herbal formula (K. C. Huang, Pharmacology of Chinese Herbs, Section II, CRC Press, Boca Raton, Ann Arbor, London, Tokyo, 1993). In other words, the combinatorial factor for making a typical four-herb formula is about 60 billion (500×499×498×497). The process of narrowing the number and kind of herb to be used in a particular formula to treat a given illness is guided by the experience of herbal doctors and the theories of TCM.

The principal herb produces the leading effects in treating the cause or the main symptom of a disease. An assistant herb helps to strengthen the effect of the principal herb and produces leading effects in the treatment of the accompanying symptoms. There are three types of adjuvant herbs: 1) those that enhance the therapeutic effects of the principal and assistant herbs or treat tertiary symptoms; 2) those that reduce or eliminate the toxicity and other side effect of the principal and the assistant herbs; and 3) those which act on complementary target tissues not specifically affected by the principal herb. A guiding herb directs the effect of other herbs to the affected site and/or coordinates and mediates the effects of the other herbs in the prescription or formulation. In contrast to most of the herbal medicines or supplements that consist of one or more parts of a single plant, the intended effects of TCM are directed at multiple tissues.

For example, a well-known TCM recipe, “Ephedra Decoction” used for treating asthma is composed of ephedra, cinnamon twig, bitter apricot kernel and licorice. Ephedra, as the principal herb, which expels cold, induces diaphoresis and facilitates the flow of the Lung Qi to relieve asthma, the main symptom. Cinnamon twig, as the assistant herb, enhances ephedra's induction of diaphoresis and warms the channels to ensure the flow of Yang Qi for reducing headache and pantalgia. Bitter apricot kernel, as the adjuvant herb, facilitates the adverse flow of the Lung Qi and strengthens the asthma relief by ephedra. Licorice as the guiding herb moderates the effects of both ephedra and cinnamon to ensure a homeostasis of the Vital Qi. While each of the four herbs clearly exhibits its respective activity, they complement as well as supplement each other when they are combined. In practice, the principal herb can be prescribed with one or more secondary herbs, depending on the symptoms at a patient's presentation (Prescription of Traditional Chinese MedicineChapter one, pp10-16, E. Zhang, editor in chief, Publishing House, Shanghai University of Traditional Chinese Medicine, 1998).

The main theories of TCM that guide the treatment of sickness with herbal medicine and other means, such as acupuncture, are 1) the theory of Yin and Yang, 2) the theory of Five Elements, 3) the theory of Viscera and Bowels, 4) the theory of Qi, Blood and Body Fluid, and 5) the theory of Channels and Collaterals.

In TCM, the first important aspect of making the proper diagnosis is to ascertain whether the disease is Yin or Yang, the two forces which control the workings of the universe. Yin represents the feminine side of nature, encompassing darkness, tranquility, depth, cold, and wetness, while Yang represents a masculine principle, encompassing light, activity, height, heat and dryness (K. C. Huang, The Pharmacology of Chinese Herbs, Second Edition. Page 2. 1999, CRC Press). Yin is commonly interpreted to be a negative force, while Yang represents a positive force. The two forces are complementary, and neither can exist without the other. Thus, TCM attempts to achieve a balance between Yin and Yang. For example, those patients who have a fever, are thirsty, constipated, or have a rapid pulse condition are of Yang character. Those individuals who have an aversion to cold, are not thirsty, have diarrhea, and a slow pulse condition are of Yin character. The property, flavor, and function of herbs can also be classified according to Yin and Yang theory. For example, herbs of cold and cool nature belong to Yin, while herbs which are warm and hot in nature belong to Yang. Herbs with sour, bitter, and salty flavor belong to Yin, while herbs with pungent, sweet and bland flavor belong to Yang. Herbs with astringent and subsiding function belong to Yin, while herbs with dispersing, ascending and floating function belong to Yang. In TCM, the principals of treatment are based on the predominance or weakness of Yin and Yang. Herbs are prescribed according to their property of Yin and Yang and their function for restoring the balance of the Yin and Yang. In so doing, the benefit of treatment is achieved.

According to the theory of Five Elements, there are five basic substances that constitute the material world (i.e., Wood, Fire, Earth, Metal and Water). In TCM this theory has been used to explain the physiology and pathology of the human body and to guide clinical diagnosis and treatment. Herbal physicians have applied the laws of generation, restriction, subjugation, and reverse restriction of the Five Elements to work out many effective and specific treatment regimens, such as reinforcing Earth to generate Metal (strengthening the function of the Spleen to benefit the Lung), replenishing Water to nourish Wood (nourishing the essence of the Kidney to benefit the Liver), supporting Earth to restrict the Wood (supplementing the function of the Spleen and disperse the stagnated Liver-Qi to treat the stagnation of Liver-Qi and Deficiency of Spleen; and strengthening Water to control Wood (replenishing the essence of the Kidney-Yin (Water) to treat hyperactivity of the Liver-Fire). Specifically, the property of some herbs is assigned to each of the Five Elements for the purpose of guiding the prescription of a TCM recipe.

In TCM, the internal organs of the human body are divided into three groups: five Viscera (the Heart, the Liver, the Spleen, the Lung and the Kidney), Six Bowels (the Gall Bladder, the Stomach, the large Intestine, the Small Intestine, the Urinary bladder, and the Triple Warmer (San Jiao), the Extraordinary Organs (the Brain, the Medulla, the Bone, the Blood Vessel, the Gall Bladder, and the Uterus). In TCM, the Viscera or the Bowel are not only anatomic units, but also concepts of physiology and pathology concerning interactions among different Viscera, Bowls, and the Extraordinary Organs. For example, the Heart also refers to some of the mental functions and influence functions of blood, hair, tongue, and skin. Yin-Yang and the Five Elements influence the interactions among these Viscera, Bowels, and the Extraordinary Organs. The complexity of interplay of the theories is used to explain the pathology of diseases to which herbs are prescribed, as discussed below.

The prescription of herbal medicine in TCM starts with the diagnosis and differentiation of symptoms, which gather the characteristics of the main symptoms, pulse, and tongue's nature or coating through Four Diagnostic Methods: inspection, listening, smelling, inquiring and palpation. Then, analysis of the Eight Principles which are Yin and Yang, Exterior and Interior, Cold and Heat, Deficiency and Excess, are performed. Finally, the character and location of the disease can be diagnosed. For instance, if the main symptom of the patient is characterized by sallow complexion, emaciated body, light red tongue with white coating (inspection), dull pain of epigastric region, which may be relieved by the patient taking warm liquid (inquiring), continued moaning (listening), stomach pain relieved by physician's pressing, and manifestation of stringed and thin pulse (palpation), this suggests the Deficiency and Cold of the Mid-Jiao (Mid-Jiao means Spleen and Stomach) which should be treated by warming the Med-Jiao and dispersing the Cold. Another example, pale complexion, tired mind (inspection), soreness and weakness of the lions and knees, impotence, seminal emission, premature ejaculation (inquiring), weak speech sound (listening), intolerance of cold with cold extremities, and deep, thin and weak pulse (palpation), manifests a weakness of the Kidney-Yang which should be treated by warming and strengthening Kidney-Yang.

In TCM, it is from Qi, Blood, and Body Fluid that come the energy needed by the Viscera and Bowels, Channels and Collaterals, tissues, and other organs for carrying-out their physiological functions; and on which the formation and metabolism of Qi, Blood, and Body Fluid depend. Herbs are used to achieve the optimal balance of Qi and the Body Fluid, that balance is believed to manifest itself in the overall health and vigor of the patient (K. C. Huang, The Pharmacology of Chinese Herbs, Second Edition. Page 2. 1999, CRC Press). Prescriptions of TCM consider the herbal effects on Qi and Blood for treatments.

TCM holds that Channels, Collaterals, and their subsidiary parts are distributed over the entire body. It is through these organs that herbs exert influence on pathological targets and achieve the improvement of health. For example, ephedra acts on the Channels of the Lung and Urinary Bladder so as to induce sweat for reliving asthma and promoting diuresis. As noted above, clinical applications of acupuncture are also guided by the theory of Channels and Collaterals.

In summary, an herb exhibits one of the Four Qi (cold, hot, warm or cool) and one of Five Flavors (bitter, acid, sweet, spicy or alkaline). The Qi or Flavor in turn exhibits distinct pharmacological properties (in Zhong Hua Ben Cao, Chief Editor: X. M. Hu, Chapter 10, Shanghai Scientific Publishing House, China, 1996). While the nature or property of each herb in TCM may be assigned as Yin or Yang, and to one of the five Elements, they act through Channels and Collaterals and are mediated via Qi, Blood and fluid to yield therapeutic effects on targets, such as Viscera and Bowels. Pathogenic factors may be disguised as decoy through the very same system of Channels and Collaterals to adversely affect the functions of Viscera and Bowels and thus cause sickness.

The Patenting of Herbal Compositions

U.S. Patents have been issued for herbal compositions used for the treatment of various diseases and other health-related problems afflicting mammals, including humans. For example, herbal compositions that include Paeonia suffuticosa have been found useful for treating viral infections, including infection from herpes and polio virus (U.S. Pat. No. 5,411,733).

Ocular inflammation can be treated with a pharmaceutical composition containing the plant alkaloid tetrandrine (U.S. Pat. No. 5,627,195). U.S. Pat. No. 5,683,697 discloses a pharmaceutical composition having anti-inflammatory, anti-fever, expectorant or antitussive action, wherein the composition includes plant parts from the species Melia, Angelica, Dendrobium, Impatiens, Citrus, Loranthus, Celosia, Cynanchum, and Glehnia. An herbal formulation comprising extracts of the roots, rhizomes, and/or vegetation of Alpinia, Smilax, Tinospora, Tribulus, Withania, and Zingiber has been found to reduce or alleviate the production of proinflammatory cytokines (U.S. Pat. No. 5,683,698). Compositions containing talc, silkworm excrement and the ingredients of twelve different herbs have been shown to be effective in reducing inflammation, pain and fever in mammals (U.S. Pat. No. 5,980,628).

Patents have also been issued for herbal compositions which find use in the treatment of cancer and cancer-related health problems. For example, U.S. Pat. No. 5,437,866 discloses a composition comprising a mixture of herbs, including species of Scutellaria barbata, as well as their extracts, which is used to ameliorate the efforts of malignancy in humans. U.S. Pat. No. 5,665,393 discloses various herbal compositions which include Glycyrrhiza glabra L. and Scutellaria baicalensis Georgi, Rabdosia rubescens, and Serenoa repens for the treatment of prostate carcinoma. Further, antitumor herbal compositions include Astragali radix, Paeonia radix, Cinnamomi cortex, Rhemannia radix and Glycyrrhizae radix for use in increasing antitumor activity of mitomycin D and doxorubicin (U.S. Pat. No. 4,613,591 and U.S. Pat. No. 4,618,495).

Acute Intracerebral Hemorrhage

Cerebrovascular disease is the most common cause of neurologic disability in Western countries, where most cerebrovascular illness is associated with atherosclerosis, hypertension, or a combination of both. The major specific types of cerebrovascular disease are (1) cerebral insufficiency due to transient disturbance of blood flow or, rarely, to hypertensive encephalopathy; (2) infarction, due either to embolism or to thrombosis of the intra- or extracranial arteries; (3) hemorrhage, including hypertensive parenchymal hemorrhage and subarachnoid hemorrhage, from congenital aneurysm; and (4) arteriovenous malformation, which can cause symptoms either of a mass lesion, infarction, or hemorrhage (C. S. Kase & L. Caplan, Intracerebral Hemorrhage, Butterworth-Heineman, 1994).

Intracerebral hemorrhage usually results from a rupture of an arteriosclerotic vessel either long exposed to arterial hypertension or made ischemic by local thrombus formation, of these two causes, hypertensive cerebral hemorrhage is usually large, single, and catastrophic. Acute intracranial hemorrhage (AICH) is a major cause of death (C. S. Kase & L. Caplan, Intracerebral Hemorrhage, Butterworth-Heineman, 1994). Current therapies for AICH are of limited efficacy and are similar to those for ischemic stroke, excluding addition of anticoagulants. Such therapies include airway maintenance, adequate oxygen, and administration of intravenous (IV) fluids to maintain nutritional and fluid intake. Also, there must be attention to bladder and bowel function. While various modalities are being tested for improved survival and clinical outcome (e.g., antioxidants and N-menthyl-D-aspartate), surgical evacuation is often the only lifesaving recourse. Nevertheless, for any treatment to be effective, such that brain damage is minimized, therapy must occur very soon after onset of symptoms.

Since multiple components of body, cardiovascular system, neural tissues, and connective tissues are involved in the cause of healing of AICH (J. M. Henry et al., Stroke, Third Edition, Churchill Livingston, 1999), one of the most promising modalities includes administration of botanical drugs. That these drugs offer curative effects to the treatment of AICH, may be attributable to natural cocktail drug efficacy which simultaneously affects multiple organs (see above). However, in attempts to emulate Western approaches, single component herbal extracts such as Ligusticum chuangxiong and ginsenosides have been studied for their usefulness in treating cerebral ischemia reperfusion (Chu et al., [Chinese J of Pharmacol] Chung Kuo Yao Li Hsueh Pao (1990) 11(2): 119-123), protection of hippocampal neurons (Lim et al., Neurosci. Res. (1997) 28(3): 191-200), treating reversible focal brain ischemia (Zhang et al., Chinese J. Pharmaceutics (1996)17(1): 44-48), treatment of ischemic stroke (Chen et al., Chin. Med. J. (1992) 105(10): 870-873), cerebral ischemia (Morishita et al., Nippon Yakurigaki Zasshi (1991) 98(6): 435-442) and acute cerebral infarction (Chen D., Chinese J. Integrated Traditional Chinese & Western Med (1992) 12(2): 71-73). In fact the use of Ligusticum chuangxiong and/or ginseng in combination with other extracts have also been tested. For example, combinations comprising Ligusticum and ginseng have been used for stroke related events (U.S. Pat. No. 4,708,949 and 4,795,742 and Lu et al.,J Pharm Pharmocol (1997) 49(11): 1162-1164); compositions comprising Ligusticum and rhubarb (Rheum palmatum) for treatment of cerebral apopjexy (U.S. Pat. No. 5,942,233). Multiple component herbal extracts have also been used for treatment of cerebral thrombosis (Zhao et al., Chinese J. Integrated Traditional Chinese & Western Med (1994) 14(2): 71-73) and cerebrovascular and brain related diseases in general (U.S. Pat. No. 5,589,182; China Patent Nos. CN 1177487 and CN 1117384).

Table 1 summarizes herbal formulations in TCM used to treat AICH.

Guo (J Chengdu College Traditional Chinese Med., 1993, Vol.16(1): 37-41) reported that the herbal composition called Fu Yuan Xing Nao Kou Fu Yie-1 reduced the intracranial pressure (ICP) and motality of experimental animals. The formula contains more than five herbs, including three that are quite toxic (in Zhong Hua Be Cao, Chief Editor: X. M. Hu, Shanghai Scientific Publishing House, China, 1996). The paper does not identify all of the herbs in the formulation nor does it provide any information on the proper herbal ratios to use in preparing the composition.

Z. K. Guo (Shanxi J. Traditional Chinese Med. 1993, Vol. 14(12): 564-565) reported that the herbal composition called Fu Yuan Xing Nao Kou Fu Yie-2 decreased ICP in patients with Acute Intracranial hemorrhagic stroke. This herbal composition contains eight component herbs that are distinct from those of Fu Yuan Xing Nao Kou Fu Yie-1. Several of these herbs are quite toxic (in Zhong Hua Ben Cao, Chief Editor: X. M. Hu, Shanghai Scientific Publishing House, China, 1996).

The herbal composition Zhuyu Huatan Decoction-1 was reported by W. Z. Yang et al. (J. Traditional Chinese Med., 1996, Vol. 37(11): 670-672; W. Z. Yang et al., Journal of Beijing University of TCM, 1997, Vol. 20(1): 8-10; W. Z. Yang et al., Chinese TCM & Western Medicine in Critical Care, 1999, Vol. 6(10): 451-453). The papers stated that the herbal preparation enhanced the resorption of hematoma and immune functions in patients with Acute Intracranial hemorrhagic stroke. This herbal composition contains more than seven herbs, including some that are quite toxic (in Zhong Hua Ben Cao, Chief Editor: X. M. Hu, Shanghai Scientific Publishing House, China, 1996). The paper did not identify all of the herbs nor did it provide the ratio of each of the botanical herbs.

W. Z. Yang et al., (Chinese J. Integrated Traditional Chinese & Western Med., 1996, Vol. 16(2): 87-89) reported that the herbal composition Zhuyu Huatan Decoction-2 reduces the serum IgG and lymphocyte proliferation rate in patients with Acute Intracranial hemorrhagic stroke. The senior author is also an author of the paper discussed above. Presumably the formulas cited in the two papers are similar since a simular formula name is used in both papers. However, this paper only disclose five component herbs; each of which is also one of the seven herbs disclosed in Zhuyu Huatan Decoction-1.

W. Z. Yang and Z. M. Wan (Journal of Beijing University of TCM, 1996, Vol. 19(3): 64-67) reported that the herbal composition Zhuyu Huatan Decoction-3 was effective in modulation of EEG in experimental rats. The senior author is also an author of the papers discussed above. Presumably the formula cited in the papers are similar since a simular formula name is used in all papers. This paper discloses that at least six of the component herbs are the same as those used to prepare Zhuyu Huatan Decoction-1.

W. Z. Yang et al., (Journal of Beijing University of TCM, 2000, Vol.23(2): 59-60) reported that the Zhuyu Huatan Decoction-4 (not included in the Table 1) corrected cerebral cell's anoxic state after cerebral hemorrhage, and improved hypoxic tolerance. However they did not disclose the content of the formula.

S. H. Chen et al. (J. of Emergency Syndromes in Chinese Medicine, 1993, Vol. 2(6): 243˜244; and J. of Emergency Syndromes in Chinese Medicine, 1995, Vol. 4(2):58˜62) reported that Zhuyu Huatan Decoction-4 could treat AICH without any toxic and side effect on people through clinical and experimental study. They did not disclose the content of the formula.

G. J. Huang et al. (Traditional Chinese Drug Research & Clinical Pharmacology, 2000, Vol. 11(4), 219˜220) reported that Fuyuan Xingnao Decoction could markedly lower the intracranial pressure persistently. They did not disclose the content of the formula either.

X. Y. Zhang et al. (J. of Emergency Syndromes in Chinese Medicine, 1999, Vol. 8(4): 148˜149) reported that Zhongfeng Xingnao Decoction had broad prospects for treating AICH. They did not disclose the content of the formula either.

W. Z. Yang et al. (J-Tradit-Chin-Med., Mar 20, 2000; (1): 3-9) reported that Zhu Yu Hua Tan Tang can decrease the intracranial pressure both in ICH patients and experimental rabbits. The intracranial pressure-lowering effect of Zhu Yu Hua Tan Tang, though slow, is smooth and long-lasting without any rebound phenomenon, as compared to those of mannitol. They did not disclose the content of the formula either.

The present application discloses the formulation of a new botanical drug, PHY828, which is distinct in many aspects from those published in the patent or scientific literature. Components of PHY828 and TCM herbal formulas disclosed above are shown in Table 1.

The development of botanical formulation of PHY828 comprising four botanicals is based on the theory of traditional Chinese medicine. As stated above, the combinatorial factors for making a typical four-herb comprising formula are about 60 billion (500×499×498×497). Moreover, given the number of combinations that must be eliminated to achieve a new efficacious herbal formula (i.e., 1 out of 60 billion), the narrowing down process based on the skill of the practitioner and theories of TCM represents an inventive step.

TABLE 1
Herbs Used in Formulas

| | | Fu Yuan | Fu Yuan | Zhuyu | Zhuyu | Zhuyu | Zhongfeng |
|---|---|---|---|---|---|---|---|
| Latin Name | | Xing Nao | Xing Nao | Huatan | Huatan | Huatan | Xingnao |
| of Herb | PHY828 | Yie-1 | Yie-2 | Decoction-1 | Decoction-2 | Decoction-3 | Decoction |
| Ligusticum | ** | ** | none | none | none | none | |
| chuanxiong | | | | | | | |
| Hort | | | | | | | |
| Panax | ** | | **10 gm | ** | ** | ** | |
| ginseng | | | | | | | |
| Panax | ** | ** | | ** | | | |
| notoginseng | | | | | | | |
| (Burk) | | | | | | | |
| F. H. Chen | | | | | | | |
| Rheum | ** | | **15 gm | ** | ** | ** | |
| palmatum L. | | | | | | | |
| Aconitum | | ** | | ** | ** | ** | |
| carmichael | | | | | | | |
| or lateralis | | | | | | | |
| Hirudo | | | **10 gm | ** | ** | ** | |
| nipponica or | | | | | | | |
| Whitmania | | | | | | | |
| pigra | | | | | | | |
| Pinellia | | ** | | ** | ** | ** | |
| pedatisecta or | | | | | | | |
| Arisaema | | | | | | | |
| heterophyllum | | | | | | | |
| Erphorbia | | | **10 gm | | | | |
| kansui | | | | | | | |
| Salvia | | ** | | ** | | ** | |
| miltiorrhiza | | | | | | | |
| or Salvia | | | | | | | |
| przewalskii | | | | | | | |
| Alisma | | | **15 gm | | | | |
| plantago-aquatica | | | | | | | |
| Uncaria | | | **15 gm | | | | |
| rhynchophylla | | | | | | | |
| or Uncaria | | | | | | | |
| sinensis | | | | | | | |
| or Uncaria | | | | | | | |
| macrophylla | | | | | | | |
| Acorus | | | **12 gm | | | | |
| calamus | | | | | | | |
| or Acorus | | | | | | | |
| tatarinowit | | | | | | | |
| Other herbs | | ** | | ** | ** | ** | ** |
| undefined | | | | | | | |
| Total number | 4 | >5 | 8 | >7 | >5 | >6 | |
| of herbs in | | | | | | | |
| the formula | | | | | | | |
| Ratio of herbs | yes | none | yes | none | none | none | |
| disclosed | | | | | | | |

Note:
**means herb used in the formula.

### SUMMARY OF THE INVENTION

The present invention provides herbal compositions comprising PHY828 for the treatment of one or more cerebral vascular disorders or diseases. Preferably, the compositions comprise materials from plant species of the following genera of herbs: one species of Ligusticum, two different species of Panax and one species of Rheum. More preferably, the plant species are Ligusticum chuanxiong Hort, Panax ginseng, Panax notoginseng and Rheum palmtum.

In one embodiment of the invention, the compositions comprise said herbs with a range of dried weight ratio for each herb of about 1 to 10:1 to 10:1 to 10:1 to 10, respectively. In a preferred embodiment, the compositions comprise the herbs with a dried weight ratio for each herb of about 2:2:2:1, respectively.

In one aspect of the invention, the plant species Panax ginseng is replaced with Codonopsis pilosula(Franch) Nannf, Pseudostellaria heterophylla (Miq.) Pax ex Pax et Hoffin. [P. rhaphanorhiza(Hemsl.) Pax] and/or Panax quinquefolium L. In another aspect of the invention, Ligusticum chuanxiong Hort can be displaced by Ligusticum chuanxiong Hort. cv. Fuxiong, and/or Ligusticum officinale (Makino) kitag. [Cnidium officinale Makino].

The present invention provides the compositions in an ingestible form or an injectible form. The ingestible form is selected from the group consisting of a powder, solution, capsule, and tablet. Additionally, the compositions of the present invention further comprise a pharmaceutically acceptable carrier.

Also provided by the present invention are methods of making the compositions comprising PHY828. In one embodiment of the invention the method comprise:

a) mixing and boiling in water, plant materials from Ligusticum and Panax to obtain a solution.

b) boiling separately in water, plant materials from Rheum to obtain a solution;

c) cooling, filtering, and sterilizing the solutions; and mixing the sterilized solutions together to obtain the composition.

In another embodiment of the invention, the method comprises:

a) mixing and boiling in water plant materials from Ligusticum, and Panax to obtain a solution;

b) adding plant materials from Rheum to the solution in step a) at about the last 10-15 minutes of boiling;

c) cooling, filtering and sterilizing the solution to obtain the composition.

In another embodiment of the invention, the method comprises:

preparing an extract with water from two different plant species of Panax, from one plant species of Ligusticum, and from one plant species of Rheum to obtain the composition.

In another embodiment of the invention, the method comprises:

a) preparing an extract from two different plant species of Panax comprising boiling plant materials from Panax in alcohol to obtain an extract and filtering and concentrating the extract;

b) preparing an extract from one plant species of Ligusticum comprising boiling plant material from Ligusticum in water for steam distillation; collecting condensate from the steam distillation; adding surfactant to the condensate to obtain an aqueous solution; and filtering and concentrating the aqueous solution to obtain an extract;

c) preparing an extract from one plant species of Rheum comprising boiling in water, plant materials from Rheum;

d) mixing the three extracts together; sterilizing the extract to obtain the composition.

In another embodiment of the invention, the method comprises preparing an extract with organic solutions from two different plant species of Panax and from one plant species of Ligusticum and preparing an extract with water from one plant species of Rheum to obtain the composition.

The plant materials from Rheum are boiled separately in water preferably for about 10 to 15 minutes or by adding to the boiling of the plant materials from Panax and Ligusticum during about the last 10-15 minutes of boiling. Extractions are carried out with the plant materials from Panax and Ligusticum by boiling or reflux for preferably about one to two hours.

The herbal compositions of the present invention are useful for treating cerebral vascular disorder selected from the group consisting of head injuries, ischemic stroke, hemorrhagic stroke, intracerebral hemorrhage, intracranial infarction and vascular dementia. In a preferred embodiment of the invention, the PHY828 composition is used to treat acute intracerebral hemorrhage, ischemic stroke, or hemorrhagic stroke.

The present invention provides methods for treating cerebral vascular disorder by administering to a mammal in need thereof, a therapeutically effective amount of a composition of PHY828. The treatment could be administered up to about 72 hours after the onset of a cerebral vascular disorder. Preferably, the methods are used to treat mammals with head injuries, acute intracerebral hemorrhage, ischemic stroke, or hemorrhage stroke. Most preferably, the methods of the present invention are used to treat human patients (Homo sapiens) diagnosed with acute intracerebral hemorrhage, ischemic stroke, or hemorrhagic stroke.

The methods of the present invention further comprise monitoring the improvement of the mammal by obtaining indices based on quality of life endpoints. The indices are selected from the group consisting of ability of returning to an independent lifestyle, ability of returning to work and a reduction in the cumulative points of residual neural functional impairment. Additionally, the methods of the present invention further comprise monitoring the improvement of the mammal by obtaining indices based on mortality rate.

Moreover, the present invention provides a composition consisting essentially of materials from plant species of the following genera of herbs: one species of Ligusticum, two different species of Panax, and one species of Rheum. Preferably, the plant species are Ligusticum chuanxiong, Panax ginseng, Panax notoginseng and Rheum palm atum.

Further, the present invention provides a composition consisting of material from plant species of the following genera of herbs: one species of Ligusticum, two different species of Panax, and one species of Rheum. Preferably, the plant species are Ligusticum chuangxiong, Panax ginseng, Panax notoginseng and Rheum palmatum.

### DETAILED DESCRIPTION OF THE PREFERRED EMBODIMEMTS

I. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Decoction: Decoction is usually prepared by boiling the botanical materials that contain water soluble and heat stable constituents for a period of time.

Mass effect: On image analysis, mass effect means the mass of a pathological tissue that compresses the neighboring tissues or organs, e.g., in AICH, if a hematoma becomes so large, it causes brain midline drift or compression of the neighboring cerebral ventricle, etc.

Neural Functional Impairment: it is evaluated by the standardized scales, e.g., National Institutes of Health Stroke Scale (NIHSS), including level of consciousness, gaze, motor function, facial paresis, dysarthria, or sensory, etc.

Herb. Technically speaking an herb is a small, non-woody (i.e., fleshy stemmed), annual or perennial seed-bearing plant in which all the aerial parts die back at the end of each growing season. Herbs are valued for their medicinal, savory or aromatic qualities. As the word is more generally used and as the word is used herein, an “herb” refers to any plant or plant part which can be used as food supplement, medicine, drug, or for any therapeutic or life-enhancing purposes. Thus, as used herein, an herb is not limited to the botanical definition of an herb but rather to any botanical, plant or plant part used for such purpose, including any plant or plant part of any plant species or subspecies of the Metaphyta kingdom, including herbs, shrubs, subshrubs, and trees. Plant parts used in herbal compositions include, but are not limited to, seeds, leaves, stems, twigs, branches, buds, flowers, bulbs, corms, tubers, rhizomes, runners, roots, fruits, cones, berries, cambium, and bark.

Herbal Composition. As used herein, an “herbal composition” refers to any composition which includes herbs, herbal plants, herbal plant parts and/or herbal extracts. Thus, as used herein, an herbal composition is any herbal preparation, including herbal food supplements, herbal medicines, herbal drugs and medical foods. Examples of herbal compositions include, but are not limited to, the following components: a whole plant or a plant part of a single plant species; whole plants or plant parts of multiple plant species; multiple components derived from a single plant species; multiple components derived from multiple plant species; herbal extracts; or any combination of these various components.

For a thorough review of various herbal compositions, see, for example, Kee Chang Huang, The Pharmacology of Chinese Herbs, CRC Press (1993), herein incorporated by reference in its entirety.

Extracts. As used herein, the term “extract” refers to a concentrated preparation of a vegetable or animal drug obtained by extracting the active constituents therefrom with a suitable solvent; in some instance, evaporating all or nearly all the solvent and adjusting the residual mass or powder to a prescribed standard. Extracts are prepared in three forms, semiliquid or of syrupy consistency, pilular or solid, and as dry powder.

In a related aspect, extracts can be considered solutions of active ingredients obtained by soaking or steeping preparations of vegetable or animal crude drugs in liquids (maceration) at high temperature or by passing such crude drugs through porous substances (percolation) for use as a medicinal agent. Further, medicinal agents of this type may be in the form of tinctures or fluid-extracts [sic] (Remington's Pharmaceutical Sciences, 1985).

In one embodiment, extracts are concentrated forms of crude drugs used in a variety of solid and semisolid dosage forms (in Remington's Pharmaceutical Sciences 17^{th} ed. Gennao, ed, Chapter 84, pp. 1516-1517, Mack Publishing Co, Easton, Pa.(1985)). For example, pilular (i.e., plastic masses) extracts are of a consistency where they are suitable for pill masses and are made into pills (e.g., pure Glycyrrhiza extract USP). Further, pilular masses are well suited for use in ointments and suppositories. Powdered extracts are better suited for powdered formulations such as capsules, powders and tablets. Further, semiliquid or extracts of syrupy consistency can be used in the manufacture of pharmaceutical preparations (Remington's Pharmaceutical Sciences, 1985).

In another embodiment, the extract is in fluid-extract [sic] form. For example, fluid-extracts include, but are not limited to, liquid preparations of vegetable drugs comprising alcohol as the solvent or as a preservative, or both, where traditionally each ml contains the therapeutic constituents of 1 gram of the drug that it represents. Fluid-extracts [sic] can be made by percolation as a general method (Remington's Pharmaceutical Sciences, 1985).

In one embodiment, the extract is in tincture form. For example, tinctures may include, but are not limited to, alcoholic or hydroalcoholic solutions prepared from vegetable matter or form chemical substances. Tinctures may be made by either percolation or maceration and are traditionally assigned potency by the amount of activity of a specified weight of the drug (in grams) per 100 ml of tincture (Remington's Pharmaceutical Sciences, 1985). For example, Sweet Orange Peel Tincture contains 50 g of sweet orange peel per 100 ml of tincture.

The amount of an herbal medicine or a botanical drug (Guidance for Industry: Botanical Drug Products, Aug. 10, 2000, published by Food and Drug Administration of the United States of America) administered to patients can be expressed in different units. For example, it may be expressed in grams of raw herbs per kilogram of body weight of a patient per time interval; the weight in grams usually means the dried weight of all herbs that constitute a multi-component formula. Or it may be expressed in milliliters of a decoction or an herbal preparation; in this case, the amount of raw herbs in grams that were used to prepare a milliliter of decoction or an herbal preparation is specified. Or it may be expressed in grams of herbal medicine; in this case, one milliliter of decoction or an herbal preparation administrated may weigh slightly over one gram since the specific gravity of an herbal preparation is usually greater than 1.0. (Commonly Used Chinese Herbal Formulas with Illustrations, H-Y Hsu and C-S Hsu, 1980, Oriental Healing Institute, Los Angeles, USA).

Using the PHY828 preparation as an example, when the preferred formula ratio is 2:2:2:1, one milliliter of the PHY828 preparation contains 0.7 grams of dried weight of four raw herbs. In the regimen, when 30 ml of the PHY828 preparation is administered to a patient four times a day, this means that 21 grams of dried weight of four raw herbs in 2:2:2:1 ratio were used to prepare the 30 ml dose. The 30 ml dose may weigh over 30 grams since the specific gravity of the PHY828 preparation is slightly greater than 1.0.

Stroke, as used herein, is a condition due to the lack of oxygen to the brain which may lead to reversible or irreversible paralysis. Further, the damage to a group of nerve cells in the brain is often due to interrupted blood flow, caused by a blood clot or blood vessel bursting. Depending on the area of the brain that is damaged, a stroke can cause coma, paralysis, speech problems and dementia. In a related aspect, strokes can be classified by ischemic or hemorrhagic syndromes, where the former is defined by cerebrovascular disorders caused by insufficient cerebral circulation and the latter by bleeding into the brain tissue or meningeal spaces. In a preferred embodiment, the present invention provides treatment methods for both mortality rate and Quality of Life (Stroke, Chapter 18, in Parmacotherapy (Dipiro et al., eds) 4^{th} ed. 1999, Appleton and Lance, USA.).

Importance of Component Ratios of Herbs in an Herbal Formula. It is well known in the art that the ratio or proportion of component herbs in an herbal formula is very important for achieving the desired therapeutic effects. Alteration of the ratio of the same herbal formula can be used to treat distinct illness.

The addition or deletion of a component herbs from a formula could drastically alter targeted indications (Commonly Used Chinese Herbal Formulas with Illustrations, Part I, H-Y Hsu and C-S Hsu, 1980,Oriental Healing Institute, Los Angeles, USA). The novelty of PHY828 lies in the selection of the four primary herbs used in the composition as well as the use of these herbs in the preferred ratios of the herbs (Table 2) so that they work synergistically to reduce significant undesirable side effects while delivering the efficacy.

Importance of Pre-Treating Raw Herbs Prior to Storage and Production. Some raw herbs are pre-treated with dry heat, water system, fermentation, vinegar, etc. the purpose of such pre-treatment are to reduce toxicity, enhance efficacy and bioavailability, for long term storage, etc. (Zhong Hua Ben Cao, Chapter 7, Chief Editor: X. M. Hu, Shanghai Scientific Publishing House, China, 1996). The ginseng used in PHY828 was pre-treated with steam, hence called “red ginseng”. However, dried ginseng (“white ginseng”) may also be used in PHY828.

Mortality Rate. The mortality rate as used herein is the proportion of deaths in a population suffering a sickness or in a specific group number of the population, where mortality is defined as the death rate or ratio of the number of deaths to the total number of sick in the population. For example, the 30 day mortality rate after ischemic stroke symptom onset can vary from about 13.3% in ischemic stroke (e.g., after treatment with tissue type plasminogen activator, see Albers et al., JAMA, 2000, 283(9):1145-1150) to greater than about 65% (e.g., hemorrhage stroke, see Mahaffey et al., Am Heart J, 1999,138(3 Pt 1):493-499).

Quality of Life. Quality of life (QOL) refers to the general well-being of an animal, especially a mammal, even more specifically a human. The QOL of an individual can be evaluated based on any one parameter, a group of two or more parameters or on a general overall evaluation or score. Example of useful indices for evaluating QOL include, but are not limited to those associated with sleeping patterns; eating patterns; drinking patterns; agility; mobility; skin tone; vision; hair retention/loss/growth; muscle tone; muscle mass; strength; weight; sinus health; presence, absence, or degree of inflammation; feelings of discomfort; ability to accomplish specific tasks; anxiety levels; response times; ability to concentrate; memory retention; verbal ability; sound perception; presence, absence or degree of headaches; muscle spasms; nerve damage; taste; touch; smell; presence or absence of opportunistic diseases; and presence or absence of parasites.

One skilled in the art of QOL evaluations can determine whether a particular treatment appears to enhance a patient's life expectancy and quality of life (even for those patients not responding to the usual treatments). For example, effective treatments of gastrointestinal diseases may be determined by several criteria, including, but not limited to, an enteritis score (based upon a composite score of clinical symptoms such as abdominal pain, cramping, stool guaiac and diarrhea), as well as related endpoints such as percent chemotherapy dose delivered, days of hospitalization, transfusion, intravenous fluid therapy, antimotility agents, and ability to eat.

With respect to a treatment effect, the subjective symptoms of the patient do not always coincide with the result of the test conducted by the doctor. For example, even in the case where an unfavorable test result is obtained, such as when the occurrence of urinary incontinence and voiding are reduced but not significantly, the patient believes that the treatment has worked, with the result that the QOL is improved.

Baseline evaluations can be entered as part of the treatment protocol whereby various criteria are measured and correlated with QOL. Further, patients can report in a patient diary events such as feeling “fair” or experiencing “moderate” pain. These measures are then used during and after treatment to evaluate whether the patient feels that the quality of life has improved. In this PHY828 patent, improvement of QOL is evaluated by GOS (Glasgow Outcome Scale) or NIHSS (National Institutes of Health Stroke Score). (See, Science and Technology Press, Chapter 2, 1999, China, R. M. Herndon, Handbook of Neurologic Rating Scales. Demos Vermander, N.Y., 1997).

II. PHY828

Introduction. PHY828 contains at least herbs from the following plant genera: Ligusticum, Panax and Rheum. One particularly effective PHY828 composition includes the plants Ligusticum chuanxiong Hort, Panax ginseng, Panax notoginseng, and Rheum palmatum L. additional herbs or ingredients may be added to produce any particular PHY828 composition.

A preferred formulation of PHY828 is provided in Table 2-1.

TABLE 2-1
Herbal Ingredients of Preferred Formulation of PHY828

| | | Preferred |
|---|---|---|
| | | Dry Weight |
| Name | Plant parts | Ratio |
| Ligusticum chuanxiong Hort | Whole Plant, Root and/or | 2 |
| | Leaves | |
| Panax ginseng | Whole Plant, Root and/or | 2 |
| | Leaves | |
| Panax notoginseng (Burk) | Whole Plant, Root and/or | 2 |
| F. H. Chen | Leaves | |
| Rheum palmatum L. | Whole Plant, Root and/or | 1 |
| Rheum officinacle Baill | Leaves | |
| Rheum tanguticu maxim.ex Balf | | |

Various other weight to weight ratios of the primary herbs in PHY828 are also contemplated in the invention (see Table 2 and Table 3).

Substitutes in PHY828 Formula

Panax ginseng can be replaced with Codonopsis pilosula(Franch) Nannf, Pseudostellaria heterophylla (Miq.) Pax ex Pax et Hoffm. [P. rhaphanorhiza(Hemsl.) Pax], and/or Panax quinquefolium L.

Ligusticum chuanxiong Hort can be replaced with Ligusticum chuanxiong Hort. cv. Fuxiong, and/or Ligusticum officinale (Makino) kitag. [Cnidium officinale Makino].

Panax notoginseng (Burk) F. H. Chen includes Panax notoginseng (Burk.) F. H. Chen ex C. Chow [P. pseudo-ginseng Wall, var. notoginseng (Burk.) Hoo et Tseng].

Rheum includes Rheum palmatum L., Rheum palmatum L. var. tanguticum Maxim. Ex Regel [R. tanguticum Maxim. ex Balf.], and Rheum officinale Baill. (Zhong Hua Ben Cao, Chief Editor: X. M. Hu, Shanghai Scientific Publishing House, China, 1996).

Accordingly, the above mentioned herbs are also contemplated in the present invention. The dry weight ratio for each of the four herbs in the PHY828 formulation can maintain its efficacy for treating AICH within the following ranges: about 1 -10:1 -10:1 -10:1 -10 (Ligusticum chuanxiong Hort: Panax ginseng: Panax notoginseng (Burk) F. H. Chen: Rheum), and preferably, about 1-6:1-6:1-6:1-6, respectively. If Panax ginseng is replaced with Codonopsis pilosula(Franch) Nannf, the amount of Codonopsis pilosula(Franch) Nannf in the formulation is about three to five times the dry weight of Panax ginseng in the formulation. Likewise, if Panax ginseng is replaced with Pseudostellaria heterophylla, the amount of Pseudostellaria heterophylla is about three to five times the dry weight of Panax ginseng in the formulation. If Panax ginseng is replaced with Panax quinquefolium L, the amount of Panax quinquefolium L in the formula is about one to two times the dry weight of Panax ginseng in the formulation.

Examples of the dry weight ratios of the four herbs in PHY828 formula are listed in Table 2-2.

TABLE 2-2
Examples of Dry Weight Ratio of the Four Herbs in PHY828

| | | | | | | | | | | | | | | | | | | |
|---|---|---|---|---|---|---|---|---|---|---|---|---|---|---|---|---|---|---|
| Ligusticum | 3 | 3 | 3 | 4 | 3.5 | 2 | 5 | 4 | 4.5 | 1 | 1 | 2 | 3 | 2 | 1 | 1 | 2 | 1 |
| chuanxiong Hort | | | | | | | | | | | | | | | | | | |
| Panax ginseng | 3 | 3 | 6 | 3 | 3.5 | 4 | 5 | 2 | 2 | 2 | 3 | 5 | 2.5 | 4 | 3 | 2 | 2 | 1 |
| Panax notoginseng | 3 | 2 | 2 | 2 | 2 | 4 | 5 | 3 | 2 | 4 | 2 | 1 | 3 | 3 | 3 | 3 | 2 | 1 |
| (Burk) F. H. Chen | | | | | | | | | | | | | | | | | | |
| Rheum palmatum L. | 1 | 2 | 1 | 1 | 1 | 2 | 1 | 1 | 1.5 | 3 | 4 | 2 | 1.5 | 1 | 3 | 4 | 1 | 1 |

Production of PHY828

Production of PHY828 formula can be prepared by one of the following two processes:

1) Raw herbs, except Rheum, are individually cut into small pieces and mixed together. The mixture, is boiled in water for about one hour; Rheum is cut into small pieces and boiled in water for about 10 minutes, separately or added to the other three herbs for about the last 10 minutes of boiling. The volume of water used is about 10 times of the total dry weight of herbs. The solutions as a liquid formulation are cooled, mixed, filtered, sterilized and bottled in a protective container for clinical use. Sterilization can be achieved by various means, including but not limited to high temperature treatment (Practical Pharmaceutical Technology, Shi Yong Yao Wu Zhi Ji Ji Shu, People Medicine Publishing House, Beijing, 1999, PP. 493-502).

2) Alternatively, the cut raw herbs can be boiled or extracted with water or organic solvents, individually. Rheum is boiled in water for about 10 minutes, separately. Organic solvents are removed from the extracts and reconstituted in an aqueous solution. The resultant aqueous solutions as a liquid formulation are cooled, mixed, filter, sterilized and bottled in a protective container for clinical use (Practical Pharmaceutical Technology, Shi Yong Yao Wu Zhi Ji Ji Shu, People Medicine Publishing House, Beijing, 1999, PP. 493-502)

Herbs, made according to the government regulations or specifications, are purchased from reputable commercial sources in China.

Process controls are utilized to ensure the uniformity and integrity of the product. Such process controls include, but are not limited to, checking the volume of the process liquor, HPLC determinations to establish Chemical Fingerprintings to verify identity of the raw materials, inspections and tests of intermediate and final products. Accepted Quality Level (AQL) limits are established for each conducted analysis and for each step of the manufacturing as well as production control.

All of the components used in the production process are assigned a specific lot number in the Production Batch Record. Quality control records are reviewed before a batch is released. Purified marker substances are used for identification and quality control of the raw materials as well as the herbal substances during production.

III. Pharmaceutical Formulations

The compositions of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical formulation for systemic administration according to the invention may be formulated for enteral, parenteral or topical administration. Indeed, all three types of formulations may be used simultaneously to achieve systemic administration of the active ingredient.

While individual needs vary, determination of optimal ranges of effective amounts of each component is within the skill of the art.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action.

PHY828 can be used in the form of a medicinal preparation, for example, in solid, semi-solid or liquid form which contains PHY828, as an active ingredients, in admixture with an organic or inorganic carrier or excipient suitable for external, enteral or parenteral applications. The active ingredient may be compounded, for example, with the usual non-toxic pharmaceutically acceptable carriers for tablets, pellets, capsules, suppositories, solutions, emulsions, suspensions, and any other form suitable for use. Formulations of the present invention encompass those which include talc, water, glucose, lactose, gum acacia, gelatin, mannitol, starch paste, magnesium trisilicate, corn starch, keratin, colloidal silica, potato starch, urea and other carriers suitable for use in manufacturing preparations, in solid, semisolid or liquid form and in addition auxiliary, stabilizing, thickening and coloring agents and perfumes may be used.

For preparing solid compositions such as tablets or capsules, PHY828 is mixed with a pharmaceutical carrier (e.g., conventional tableting ingredients such as corn starch, lactose, sucrose, sorbitol, talc, stearic acid, magnesium stearate, dicalcium phosphate gums and other pharmaceutical diluents or composition containing a substantially homogeneous mixture of PHY828, or a non-toxic pharmaceutically acceptable salt thereof. When referring to the preformulation compositions as substantially homogenous, it is meant that the active ingredients are dispersed evenly throughout the composition so that the composition may be readily subdivided into equally effective unit dosage forms such as tablets, pills and capsules. This solid preformulation composition is then subdivided into unit dosage forms of the type described above containing an effective amount of the composition of the present invention, preferably in capsules.

The tablets or pills containing PHY828 can be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coating such materials including a number of polymeric acids and mixtures of polymeric acids with such materials as shellac, cetyl alcohol and cellulose acetate.

The liquid forms, in which PHY828 may be incorporated for administration orally or by injection, include aqueous solution, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin.

Liquid preparations for oral administration may take the form of, for example, solutions, syrups or suspensions, or they may be presented as a dry product for reconstitution with water or other suitable vehicles before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

For buccal administration, the compositions of the present invention may take the form of tables or lozenges formulated in conventional manners.

PHY828 may also be formulated for parenteral administration by injection, which includes using conventional catheterization techniques or infusion. Formulations for injection may be presented in unit dosage form, e.g., in ampules, or in multi-dose containers, with an added preservative. The compositions may take such form as suspensions, solutions or emulsions in oily or aqueous vehicles, and may contain formulating agents such as suspending, stabilizing, and/or dispersing agents. Alternatively, the active ingredients may be in powder form for reconstitution with a suitable vehicle, e.g., sterile pyrogen-free water, before use.

Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

In practicing the methods of this invention, PHY828 may be used alone or in combination, or in combination with other therapeutic or diagnostic agents. In certain preferred embodiments, the compounds of this invention may be coadministered along with other compounds typically prescribed for these conditions according to generally accepted medical practice.

In practicing the methods of this invention, PHY828 may be used alone or in combination, or in combination with other therapeutic or diagnostic agents. In certain preferred embodiments, the combination with other therapeutic or diagnostic agents. In certain preferred embodiments, the compounds of this invention may be coadministered along with other compounds typically prescribed for these conditions according to generally accepted medical practice. The compounds of this invention can be utilized in vivo, ordinarily in mammals, such as humans, sheep, horses, cattle, pigs, dogs, cats, rats and mice, or in vitro.

Actual methods for preparing administrable compositions and adjustments necessary for administration to subjects are known or apparent to those skilled in the art and are described in more detail in, for example, Remington's Pharmaceutical Science, 15^{th} Ed., Mack Publishing Company, Easton, Pa. (1980), which is incorporated herein by reference.

“Therapeutic index” is used to designate a qualitative statement of the selectivity of a drug when a therapeutic and an untoward effect are being compared. For example, if the untoward effect is designated as T (for toxic) and the therapeutic effect as E (for efficacy), the therapeutic index may be defined as TD50/ED50 or a similar ratio at some other arbitrary levels of response.

IV. Pharmacology and Toxicology Studies in Animals

The present invention is based in part on the finding that PHY828 is useful for treating cerebral disorders in animals. Specifically, in the pharmacological studies performed with animals, it was found 1) PHY828 herbal medicine significantly lowered experimentally induced intracranial pressure (ICP) in rabbits; 2) PHY828 alleviated cerebral edema and promoted the absorption of hematoma in rats; 3) PHY828 stimulated the regeneration of neurocytes in rats; and 4) PHY828 improved the hemorheology in rats.

The present invention is also based in part on the finding that PHY828 exhibits low toxicity in both acute and long-term toxicity tests. Specifically, in an acute toxicity test in mice, it was found that PHY828 has an LD₅₀ of 223.82±20.89 g/kg. This dose is 134 times greater than that given to human subjects. It was also found that it was safe to give SD rats PHY828 at an oral dose of 83.5 g/kg/day for three months. This dose is 50 times great than the dose given to human subjects.

### EXAMPLES

A. Animal Studies

1. Pharmacology of PHY828

High intracranial pressure (ICP) and brain edema were experimentally induced in rabbits. PHY828 at a dose of about 20 gm-unit*/kg body weight was administered to rabbits via buccal route (*a gram-unit is defined as amount of raw herbs used for the preparation of the composition). The ICP began decreasing (32 to 56% reduction) one hour after the rabbits (N=21) started receiving PHY828 and reached its lowest level two hours after receiving PHY828. In contrast to control rabbits (N=21) treated with mannitol, there was no rebound phenomenon of ICP with PHY828. Additionally, osmotic pressure did not increase in the rabbits treated with PHY828 in contrast to the rabbits treated with mannitol.

PHY828 at a dose of about 25 gm-unit*/kg body weight was administered orally daily for ten days to rats (N=20) with an experimentally induced hematoma. PHY828 was able to alleviate cerebral edema and promote the absorption of hematoma.

PHY828 at a dose of about 25 gm-unit*/kg body weight was administered orally daily for ten days to rats (N=20) with collagen induced hematoma. In this model, PHY828 stimulated the regeneration of neurocytes in rat. The nissl body of neurocytes in the hippocampus zone was found to be increased. PHY828 was shown protection to the brain cells, and invigorates the neurocytes vital activity and function

PHY828 at a dose of about 25 gm-unit*/kg body weight was administered to rats (N=20) with the experimentally induced abnormal hemorheolgy. PHY828 improved the hemorheolgy of the rat. The effects of PHY828 are beneficial to cerebral blood supply after supply after onset of AICH. PHY828 promotes the recovery of injured tissue or neural function.

2. Toxicology of PHY828

An acute toxicity test was performed in mice. PHY828 has an 223.82±20.89 g/kg. This dose is 134 times greater than the dose given to human subjects.

A long-term toxicity test performed with SD rats. PHY828 at an oral dose 83.5 g/kg/day was given to SD rats for three months. This dose is 50 times greater than the dose given to human subjects. PHY828 was found to be safe. PHY828 exhibits minimal toxicity and side effect in acute and long-term toxicity tests in rodents.

3. Preparation of PHY828 Formula

### Example 1

### Processing of Herbs for Preparation of PHY828

PHY828 was provided using the preferred formulation (see Table 2-1) such as: Panax ginseng about 175.5 Kg, Ligusticum chuanxiong Hort about 175.5 Kg, Panax notoginseng (Burk) F. H. Chen about 175.5 Kg, Rheum palmatum L. about 87.8 Kg. The raw herbs were cut into small pieces, weighed, mixed in stainless steel double-deck container and boiled in about 4265 liters of water for about two hours. Rheum was boiled separately for about 10 to 15 minutes in about 878 liters of water. After cooling, the liquid was filtered, sterilized at high temperature and poured into brown bottles for clinical use. All of the herbal extracts were freshly prepared.

### EXAMPLE 2

### Processing of individual Herbs of PHY828 and Preparation of PHY828

The processing of the individual herbs is described in A-C Sections below and the preparation of PHY828 using the individually processed herbs is described in D Section below.

A. The production of extract from Panax ginseng and Panax notoginseng (Burk) F. H. Chen mixture.

Panax ginseng about 175.5 kg and Panax notoginseng (Burk) F. H. Chen about 175.5 kg were placed in a multiple-effect reactor tank.

2. About 2100 liters alcohol (V/V 60%) were added to the herbs in the reactor tank, and then extracted with boiling-reflux for about 1.5 hours. The extract was filtered with a high-speed filter, and the filtrate was concentrated to a specific gravity of 1.26-1.30 at 70 degree Celsius under reduced-pressure, then stored in a refrigerator of 0 to 5 degree Celsius.

3. To the herbal residues, pure water in an amount of about 9 times the volume (V/V) of the two raw herbs from the above step was added into the reactor tank, and extracted with boiling-reflux for about 1.5 hours. The process was repeated three times. The combined extracts were filtered in a high-speed filter, and the filtrate was concentrated to a specific gravity of 1.03-1.04 at 75 degree Celsius under reduced pressure in a concentration device.

4. When the solution reached the pre-set specification, the concentrated solution is then mixed with 95% (V/V) alcohol to reach 70% (V/V) at room temperature and left to allow the precipitants to settle overnight.

The next morning, the clear phase was collected and filtered, then the filtrate was concentrated to a specific gravity of about 1.02-1.04 under reduced pressure at 70 degree Celsius and stored in a refrigerator.

B. Extraction of Ligusticum chuanxiong Hort

1. Ligusticum chuanxiong Hort about 175.5 kg previously sliced into small pieces was placed into a multiple-functional reactor tank.

2. About 1600 liter pure water was added into the multi-functional reactor tank. The herb was soaked for about 30 minutes, and the preparation was then heated to boiling for steam distillation. The condensate was collected into a separate vessel. After about 175.5 liter of condensate was collected, Tween-80 was added to the condensate to a final concentration of 0.25%. The Tween-80 solution in the storage tank was filtered and stored in cool temperatures (0-5 degree Celsius).

3. After the aqueous solution in the vessel was drained into an extract holding tank, the herbal residue left in the vessel was then repeatedly extracted three times, each time with about 8×175.5 liters of water boiled for about 1 hour. The combined aqueous solution was filtered to remove the precipitatants and suspended particulates and then concentrated at reduced pressure at 70 degree Celsius to a specific gravity of 1.12-1.14.

4. The concentrated solution was drained into another tank for precipitation by alcohol. 95% (V/V) alcohol was added into the tank to make a final concentration of 70%. The solution is left overnight to allow the precipitants to settle.

5. The next morning, the clear phase was collected, and filtered, then the filtrate was concentrated to a specific gravity of 1.12-1.14 at 70 degree Celsius under reduced pressure, and stored in a refrigerator.

C. Extraction of Rheum Palmatum L.

1. About 87.8 kg of previously sliced Rheum palmatum L. was placed into a multiple-functional reactor tank.

2. About 8×87.8 kg liters of pure water was added into the tank, Rheum palmatum L soaked for about 30 minutes, and boiled for about 10 to 15 minutes. The extraction process was repeated for two more times. The collected extract in the holding tank was filtered to remove the particulates and suspended materials presented in the extracts. The filtrate was concentrated to a specific gravity of 1.04-1.06 at 75 degree Celsius under reduced pressure.

3. The concentrate was then discharged into a precipitation tank and mixed with about 87.8 liters of 5% gelatin. The mixture was kept at 0 to 5 degree Celsius for about 12 hours to allow the precipitant to settle. The precipitant was removed by filtration and the resulting filtrate was then further concentrated to a specific gravity of 1.04-1.06 under reduced pressure at 70 degree Celsius.

4. The concentrated solution was then mixed again with 95% alcohol to reach a final concentration of 80%, and left to allow the precipitants to settle for about 12 hours.

5. The clear phase was collected, filtered, and concentrated to a specific gravity of 1.06-1.08 under reduced pressure at about 70 degree Celsius.

6. The concentrated solution was then mixed with Tween-80 to reach about 0.7% and stored in a cold room (0 5 degree Celsius) for subsequent use in preparing the final dose mixture.

D. Preparation of PHY828.

1. In a mixing tank, non condensate extract from Panax ginseng and Panax notoginseng (Burk) F. H. Chen in process (A), and from Ligusticum chuanxiong Hort and Rheum palmatum L. in processes (B) and (C) were mixed into a homogeneous solution. The pH of the solution was adjusted to about 5.8-6.0. The mixture was boiled further for about 5 minutes, and cooled to room temperature.

2. The Tween-80 condensate of Ligusticum chuanxiong Hort in process (B) was added to the mixing tank. Distilled water was then added to the tank to a final volume of about 615 liters. The solution was mixed thoroughly and left to stand overnight.

3. The next morning, the resulting solution from step 2 above was filtered and adjusted to a pH of about 5.8-6.0.

4. The solution was dispensed into protective containers, sterilized, and packaged.

### Examples 3-15

The raw herbal dry weight ratios shown in Table 3-1, were prepared by the process described in Example 1 above:

TABLE 3-1
Preparation Number

| | 2 | 3 | 4 | 5 | 6 | 7 | 8 | 9 | 10 | 11 | 12 | 13 | 14 |
|---|---|---|---|---|---|---|---|---|---|---|---|---|---|
| Ligusticum chuanxiong Hort | 2 | 3 | 3 | 3 | 4 | 3.5 | 2 | 5 | 4 | 4.5 | 1 | 1 | 2 |
| Panax ginseng | 2 | 3 | 3 | 6 | 3 | 3.5 | 4 | 5 | 2 | 2 | 2 | 3 | 5 |
| Panax notoginseng | 2 | 3 | 2 | 2 | 2 | 2 | 4 | 5 | 3 | 2 | 4 | 2 | 1 |
| (Burk) F. H. Chen | | | | | | | | | | | | | |
| Rheum palmatum L. | 1 | 1 | 2 | 1 | 1 | 1 | 2 | 1 | 1 | 1.5 | 3 | 4 | 2 |

### Examples 16-24

The raw herbal dry weight ratios shown in Table 3-2, were prepared by the process described in Example 2 above:

TABLE 3-2
Preparation Number

| | 15 | 16 | 17 | 18 | 19 | 20 | 21 | 22 | 23 |
|---|---|---|---|---|---|---|---|---|---|
| Ligusticum chuanxiong Hort | 3 | 2 | 1 | 1 | 1 | 3 | 2 | 3 | 5 |
| Panax ginseng | 2.5 | 4 | 3 | 2 | 1 | 3 | 2 | 3 | 5 |
| Panax notoginseng (Burk) | 3 | 3 | 3 | 3 | 1 | 2 | 2 | 3 | 5 |
| F. H. Chen | | | | | | | | | |
| Rheum palmatum L. | 1.5 | 1 | 3 | 4 | 1 | 2 | 1 | 1 | 1 |

### Example 25

The composition was prepared as described in Example 1. However, when Ligusticum chuanxiong Hort was replaced with Codonopsis pilosula(Franch) Nannf, the composition (Ligusticum chuanxiong Hort: Codonopsis pilosula(Franch) Nannf: Panax notoginseng (Burk) F. H. Chen: Rheum palmatum L.) was processed and prepared at a dried, weight-to-weight ratio of 2:10:2:1.

### Example 26

The composition was prepared as described in Example 1. However, when Panax ginseng was replaced with Codonopsis pilosula(Franch) Nannf, Ligusticum chuanxiong Hort was replaced with Ligusticum officinale (Makino) kitag, the composition (Ligusticum officinale (Makino) kitag: Codonopsis pilosula(Franch) Nannf: Panax notoginseng (Burk) F. H. Chen: Rheum palmatum L.) was processed and prepared at a dried, weight-to-weight ratio of 2:10:2:1.

### Example 27

The composition was prepared as described in Example 1 above, However, when Panax ginseng was replaced with Panax quinquefolium L., Ligusticum chuanxiong Hort was replaced with Ligusticum chuanxiong Hort. cv. Fuxiong. The composition (Ligusticum chuanxiong Hort. cv. Fuxiong: Panax quinquefolium L.: Panax notoginseng (Burk) F. H. Chen: Rheum palmatum L.) was processed and prepared at a dried, weight-to-weight ratio of 2:2:2:1.

### Example 28

### Clinical Study: Treat AICH with PHY828 Botanical Drug

Recruitment of Patients.

We recruited those patients who were AICH as confirmed by CT scan, which were diagnosed as moderate and serious types, and were admitted within 72 hours from stroke attack. Patients diagnosed with moderate to severe AICH, confirmed by CT Scan and admitted within 72 hours after stroke were recruited. Patients with traumatic intracranial hemostatic edema or with mild AICH were excluded. Also excluded were those with concurrent severe impairment of heart, liver, primordial function, diabetes and arthritis.

Criteria for the patient diagnosis were based on those designed at the 4^{th} National Conference of Cerebrovascular Disease of PRC which requires a: hypertension history b: AICH confirmed by CT scan (see J. Neurology, China 1996, 12; 29(6)). Further, the condition of coma (or level of consciousness) was evaluated by Glasgow Coma Scale (GCS-see Modern Neurology Progress, Science and Technology Press, 1999, Beijing).

Basic Therapy.

Nursing Procedure: The admitted patients received absolute bed rest, and were not permitted to turn over onto the bleeding side until 24 hours after the attack. The respiratory tract was kept clear, and the patient remained in a lateral position in order to drain the oral secretion as much as possible.

High oxygen flow was continuously provided to patients and, when needed, nutrition by gastrogavage was provided 24-48 hours after attack. If the patients were unable to take food, then it was necessary to infuse nutritional fluids with nasal tubing.

The water-electrolyte metabolism and acid-base balance were maintained by appropriate means in accordance with the patients' conditions.

Complications such as infection, hypertension, and restlessness etc. were treated by appropriate means (e.g., antibiotics, and diazepam, respectively).

Treatment with PHY828 Botanical Drug.

Patients received about 20-30 ml of PHY828 four times a day, for about 30 days or longer, orally or by gastrogavage after taking food. The attending physician may adjust the dose and frequency of administration during a patient's recovery. Dehydration, diuretic and cerebral cell activators were not prescribed during the course of treatment. If cerebral hernia happens, 20% mannitol was permitted administering intravenously (125 ml, 2 to 6 times per 24 hours period) to control intracranial pressure (ICP) and edema (i.e., Western medicine treatments for AICH).

Therapeutic Evaluation.

The evaluation criteria on therapeutic effect were based on QOL drawn up by National Institutes of Stroke Scale (NIHSS- Modern Neurology Progress, Science and Technology Press, 1999, Beijing) and Glasgow Outcome Scale (R. M. Herndon, Handbook of Neurologic Rating Scales, Demos Vermande, New York, 1997).

Result and Analvsis of the Clinical Study.

The therapeutic effects of PHY828 were evaluated after 15, 30, 60, and 90 days of treatment. The cumulative points of neural functional impairment (NIHSS) were calculated. The Quality of Life based on GOS were evaluated.

The patients were examined twice by CT scan during the treatment (generally, on the 15^{th} and 30^{th} days of treatment).

The results are provided in Tables 4-1˜4-3.

TABLE 4-1
General Information

| | Period | | | Blood | | | Complicate | Medical |
|---|---|---|---|---|---|---|---|---|
| | from onset | | | Pressure | Blood sugar | | Disease | History |
| Case #-ID | to hospital | Sex | Age | (mmHg) | (mmol/L) | GCS | Scale | Scale |
| 1-117862 | 72 h | M | 82 | 150/90 | 8.9 | 4 | 4 | 6 |
| 2-117450 | 0.5 h | M | 62 | 176/100 | 4.5 | 8 | 4 | 6 |
| 3-112115 | 2 h | M | 64 | 167/107 | 7.29 | 15 | 0 | 6 |
| 4-116582 | 3 h | M | 55 | 161/94 | 5.46 | 15 | 0 | 5 |
| 5-115522 | 7 h | M | 66 | 128/75 | 5.29 | 15 | 0 | 3 |
| 6-116823 | 3 h | F | 50 | 200/120 | 5.27 | 13 | 5 | 3 |
| 7-115284 | 2 h | M | 72 | 225/110 | 4.09 | 3 | 8 | 8 |
| 8-118855 | 6 h | M | 61 | 188/113 | 7.75 | 4 | 4 | 5 |
| 9-120676 | 10 h | M | 48 | 160/100 | 6.1 | 15 | 3 | 3 |
| 10-120501 | 8 h | M | 73 | 165/90 | 5.3 | 4 | 5 | 2 |
| 11-121817 | 18 h | M | 79 | 140/67 | 5.41 | 8 | 4 | 2 |
| 12-120555 | 12 h | F | 52 | 152/85 | 6.9 | 6 | 3 | 5 |
| 13-121233 | 0.5 h | M | 49 | 151.5/103 | 5.9 | 4 | 3 | 5 |
| 14-122151 | 1.5 h | M | 67 | 216/101 | 7.08 | 4 | 5 | 5 |
| 15-121753 | 72 h | M | 67 | 160/83 | 7.19 | 8 | 5 | 4 |
| 16-122331 | 7 d | M | 80 | 161/78 | 7.62 | 8 | 3 | 4 |
| 17-123404 | 3 h | M | 77 | 264/18 | 8.8 | 8 | 5 | 8 |
| 18-123021 | 1 h | M | 70 | 186/85 | 7.8 | 8 | 3 | 3 |
| 19-122151 | 1.5 h | M | 67 | 214/101 | 7.08 | 6 | 4 | 4 |
| 20-120449 | 2 h | M | 82 | 215/115 | 17.3 | 4 | 4 | 5 |
| 21-121379 | 1 h | M | 56 | 260/110 | 6.6 | 4 | 5 | 3 |
| 22-122761 | 1 h | M | 75 | 159/63 | 6.3 | 10 | 3 | 4 |
| 23-118115 | 24 h | M | 71 | 195/105 | 10.42 | 10 | 3 | 4 |
| 24-117847* | 2 h | F | 70 | 220/110 | 6.0 | 4 | 5 | 4 |
| 25-Outpatient** | 40 d | F | 60 | 170/90 | Absent | 15 | 3 | 4 |

Note:
d = days;
h = hours.
Cases 1-23 are patients with AICH, while Case 24 and Case 25 suffered intracerebral infarction (ICI), which is a subgroup of ischemic stroke.
*Case 24 suffered acute ICI of right basal ganglia, with a left basal ganglia ICI history several years ago.
**Case 25 who suffered left basal ganglia ICI, had been treated for 40 days elsewhere before receiving PHY828 botanical drug. She was also hemiplegic and unable to walk by herself before taking PHY828. After treatment with PHY828, she recovered completely without any hemiplegia and returned to her previous work.

Glasgow Coma Score(GCS)

| Eye Opening (E) | Verbal Response (V) | Motor Response (M) |
|---|---|---|
| 4 = Spontaneous | 5 = Normal conversation | 6 = Normal |
| 3 = To voice | 4 = Disoriented conversation | 5 = Localizes to pain |
| 2 = To pain | 3 = Words, but not coherent | 4 = Withdraws to pain |
| 1 = None | 2 = No words . . . only sounds | 3 = Decorticate |
| | 1 = None | posture |
| | | 2 = Decerebrate |
| | | 1 = None |
| | | Total = E + V + M |

(See Modern Neurology Progress, Science and Technology Press, 1999, Beijing)

TABLE 4-2
Results of CT Scan

| Patient #;(see | On in-hospital | | | | | | |
|---|---|---|---|---|---|---|---|
| Table 4-1) | Vol- | | Extend to | | | | |
| Hematoma | ume | | cerebral | 15^{th} days | | 30^{th} days | |
| Location | (ml) | Mass Effect | ventricle | Volume (ml) | Mass Effect | Volume (ml) | Mass Effect |
| 1-Left thalamus | 8.17 | Midline drift over 2 mm; cerebral | Yes | Mostly absorbed | Improved | Complete | Disappearance |
| | | ventricle was compressed. | | | | absorption | |
| 2-Left exterior | 16.45 | Midline drift | none | Mostly absorbed | | | |
| capsule | | | | | | | |
| 3-Left thalamus | 8.36 | None | None | Mostly absorbed | None | Complete | None |
| | | | | | | absorption | |
| 4-Right exterior | | | | | | | |
| capsule | | | | | | | |
| 5-Right lentiform | 8.8 | Midline drift over 2 mm; cerebral | None | Mostly absorbed | Improved | Complete | Disappearance |
| nucleus and | | ventricle was compressed | | | | absorption | |
| interior capsule | | | | | | | |
| 6-Right exterior | 10.53 | No midline drift; cerebral, | None | 6.52 | Improved | Complete | Disappearance |
| capsule and | | ventricle was compressed | | | | absorption | |
| putamen | | | | | | | |
| 7-pontine | 10.88 | Midline was middle; the close | None | — | — | *Complete | *Disappearance |
| | | brain cisterna was compressed; | | | | absorption | |
| | | the brain stem was extruded to | | | | | |
| | | the left side. | | | | | |
| 8-Right Exterior | 49.5 | Right lateral ventricle was | none | Partially absorbed | Improved | **Complete | **Disappearance |
| capsule | | compressed to block. | | | | absorption | |
| 9- Right Exterior | 12.5 | None | none | Partially absorbed | None | Complete | None |
| capsule | | | | | | absorption | |
| 10-Left thalamus | 5.33 | Left lateral ventricle was | None | Partially absorbed; NEW | None | Complete | |
| | | compressed | | infarction at anterior and | | absorption | |
| | | | | posterior left lateral | | | |
| | | | | ventricle | | | |
| 11-Right basal | 25.8 | Right lateral ventricle was | None | Only 5 days after onset | | | |
| ganglia | | compressed | | | | | |
| 12-Left thalamus | 9.7 | Left lateral ventricle was | yes | Partially absorption | Improved | Complete | None |
| | | compressed and midline drift | | | | absorption | |
| 13-Left Exterior | 32.6 | Midline drift to right, right lateral | None | Partially absorbed | Improved | Only 20 days | |
| capsule | | ventricle was obstructive | | | | after onset | |
| | | hydrocephalus; and left lateral | | | | | |
| | | ventricle was compressed. | | | | | |
| 14-Left basal | 29.1 | Midline drift to right, left lateral | None | Not done because of | Only 22 days | | |
| ganglia | | ventricle. Edema around | | serious condition | | in hospital. | |
| | | hemotoma. | | | | | |
| 15-Left basal | 7.41 | None | None | Not done | | Nearly | |
| ganglia | | | | | | complete | |
| | | | | | | absorption | |
| 16- Left basal | 65.6 | Midline drift to right, left lateral | None | 37.3 | Improved | Only 22 days | |
| ganglia | | ventricle. Edema around | | | | in hospital. | |
| | | hematoma | | | | | |
| 17-Right basal | 49 | Right lateral ventricle was clearly | None | Partially absorbed | Improved | Not done | |
| ganglia | | compressed; midline drift to left. | | | | | |
| 18-Left basal | 12.2 | Edema around hematoma, | None | Partially absorbed | None | Complete | None |
| ganglia | | absence of midline drift. | | | | absorption; | |
| | | | | | | New | |
| | | | | | | infarction | |
| | | | | | | occurred in | |
| | | | | | | the right | |
| | | | | | | thalamus. | |
| 19-Left basal | 29.14 | Midline drift to right; left lateral | None | Not done because of | Not done | Nearly | Serious edema |
| ganglia | | ventricle was compressed; edema | | serious condition | | complete | around |
| | | around hematoma | | | | absorption. | hematoma; |
| | | | | | | | Midline drift to |
| | | | | | | | right; left lateral |
| | | | | | | | ventricle was |
| | | | | | | | compressed. |
| 20-Left basal | 126 | Midline drift to right; left lateral | Extend to | Death | | | |
| ganglia | | ventricle was compressed | lateral | | | | |
| | | | ventricle, 4^{th} | | | | |
| | | | ventricle | | | | |
| 21-Brain stem | 6.72 | Pontocerebellar pedunculus was | None | Not done because of | | | |
| | | clearly compressed. | | serious condition. | | | |
| 22-Right basal | 26.7 | Right lateral ventricle was | Extend to | 20 | Improved | Nearly | Improved |
| ganglia | | compressed; midline drift to left. | posterior | | | complete | |
| | | | lateral | | | absorption | |
| | | | ventricle | | | | |
| 23-Left basal | 2 | None | None | Completely absorption | None | | |
| ganglia | | | | | | | |

Note
*CT scan on day 120
**CT scan on day 56.
The first CT scan (1 hour from onset) showed only 6 ml hematoma in left exterior capsule; but after 48 hours, 2^{nd} CT scan showed the first hematoma increased to 32.6 ml along with deep coma.

TABLE 4-3
NIHSS and GOS after PHY828 with AICH and ICI

| | NIHSS | | | | *GOS | | |
|---|---|---|---|---|---|---|---|
| Patient#; | Before | | | | | | |
| (see Table 4-1) | treatment | 30 days | 60 days | 90 days | 30 days | 60 days | 90 days |
| 1 | 21 | 11 | 3 | 2 | 3 | 2 | 1 |
| 2 | 23 | 3 | 2 | 0 | 2 | 1 | 1 |
| 3 | 6 | 0 | 0 | 0 | 1 | 1 | 1 |
| 4 | 8 | 0 | 0 | 0 | 1 | 1 | 1 |
| 5 | 7 | 0 | 0 | 0 | 1 | 1 | 1 |
| 6 | 13 | 0 | 0 | 0 | 1 | 1 | 1 |
| 7** | 40 | 38 | 25 | 20 | 3 | 3 | 3 |
| 8 | 43 | 30 | 17 | 10 | 3 | 2 | 2 |
| 9 | 20 | 4 | 0 | 0 | 2 | 1 | 1 |
| 10 | 30 | 4 | 3 | 3 | 2 | 1 | 1 |
| 11 | 30 | Death | | | 5(Death) | | |
| 12 | 30 | 6 | 3 | 2 | 2 | 1 | 1 |
| 13** | 40 | 20 | 14 | 10 | 3 | 2 | 2 |
| 14** | 40 | 35 | 14 | 14 | 3 | 3 | 3 |
| 15 | 20 | 10 | 9 | 8 | 3 | 2 | 2 |
| 16 | 30 | 20 | 16 | 14 | 3 | 3 | 3 |
| 17 | 40 | 35 | 35 | 30 | 4 | 3 | 3 |
| 18 | 33 | 20 | 10 | 6 | 3 | 2 | 1 |
| 19 | 40 | 26 | 20 | 10 | 4 | 3 | 2 |
| 20** | 40 | Death | | | 5(Death) | | |
| 21** | 40 | Death | | | 5(Death) | | |
| 22 | 30 | 10 | 8 | 6 | 3 | 2 | 2 |
| 23 | 12 | 2 | 0 | 0 | 2 | 1 | 1 |
| 24 | 35 | 25 | 15 | 10 | 3 | 3 | 2 |
| 25 | 10 | 0 | 0 | 0 | 1 | 1 | 1 |

**Mechanical ventilation
Note:
Glasgow Outcome Scale (GOS) (see Modern Neurology Progress, Science and Technology Press, 1999, Beijing):
1 - full recovery, leads good and independent life
2 - moderate disability, independent but has neurological or intellectual impairment
3 - severely disabled, conscious but totally dependent
4 - vegetative survival
5 - dead

Analysis of Result

The results are provided in Table 4-1-4-3. The conclusions are as follows:

1. Carlos S. Kase et. al^{[2]}report that the mortality rate of AICH is 20-56%, and about 23-73% of survivors have severe disability, (Intracerebral hemorrhage, Carlos S. Kase, Butterworth-Heinemann, 1994.). In this clinical study, 16 out of 20 patients after treatment with PHY828 led independent lives (80%). Two patients died (10%), and two patients have severe disability (11.1% in survivors) after treatment with PHY828. The data show that PHY828 exhibits significant efficacy on AICH, including reducing both mortality rate and disability rate. (see Table 5).

2. In this clinical study, the volume of 20% mannitol supplied to a patient on average was obviously decreased, which indicates that PHY828 could reduce the elevated intracranial pressure.

3. PHY828 can enhance the absorption of the hematoma, as well as the recovery of neural function.

4. PHY828 shows efficacy on AICH patients with hemorrhage at such locations as basal ganglia, cerebellar, and pontine.

TABLE 5
Comparison PHY828 with Current Reports on AICH*

| Current Reports | Mortality Rate | Severe Disability** |
|---|---|---|
| Y. F. Guo et al.^{[1]} | 67% | 40% |
| Carlos S. Kase et. Al^{[2]} | 20-56% | 23-78% |
| Z. C. Wang^{[3]} | 9.8-22.3% | 30-60% |
| PHY828 | 10% | 10% |

^{[1]}Treatment of Cerebrovascular Disease by Chinese Experts (Zhong Guo Nao Xue Guan Bing Zhi Liao Zhuan Jia Lun Ji), Chief Editor: YuFu Guo et al, Shenyang Publishing House, Shenyang, 1995.
^{[2]}Intrcerebral hemorrhange, Carlos S. Kase, Butterwoth-Heinemann, 1994.
^{[3]}Neurosurgery, Chief editor Zhongcheng Wang, Hubei Science & Technology Press, Wuhan, 1998.
It is noted that the statistical data of reports [1]˜[3] cited above were evaluated 3-6 years after onset of AICH . In contrast, the data of PHY828 clinical study were evaluated within 90 days. Therefore, the efficacy of PHY828 maybe even better than that shown in table 5, if the evaluation were performed 3-6 years post AICH onset.
*Analysis of Reasons of Patients' Death
1. Case 11, died on the 22^{nd} day from admission. The death was attributed to a premature termination of treatment due to non-medical reasons. The case was thus not included in the statistical data in Table 5.
2. Case 20, died on the 3^{rd} day after admission as a consequence of a cerebral hernia.
3. Case 21, died on the 30^{th} day after admission as a consequence of a cerebral hernia due to large hematoma in pontine.
**Analysis of Reasons of Severe Disability.
1. Case 16 did not accept PHY828 treatment until 7 days after AICH onset, which was too late to achieve optimal efficacy. The window of treatment for AICH is from onset to 72 hours post onset. The patient's hematoma was absorbed quickly upon receiving treatment with PHY828 botanical drug.
2. Case 17 & #19, were not included in Table 5 because mechanical ventilation was terminated prematurely due to non-medical reasons.

Examples of Clinical Case Reports: Case #7 with A Pontine Hemorrhage.

A 72-years-old man was admitted to the hospital because of a sudden fall 2 hours prior to admission at 11 am on Jan. 11, 2000, (Admission Number: 115284). The patient was delivered to the hospital by ambulance.

At the hospital, he presented with a confused state of mind, and incontinence of urine, but no vomiting or incontinence of stool. The CT scan showed that there was high density 2.0×2.72×4.0 cm at pontine which caused the neighboring brain cisterna to be compressed. The brain stem was extruded to the left side. During the CT scan, the patient vomited gastric contents once. The patient has a history of hypertension and has been treated with anti-hypertension drugs for over twenty years. He also has had a chronic cough with phlegm for over twenty years.

Physical Examination: Temperature was 36.7 degree Celsius; respiration rate was 19 min; blood pressure was 225/110 mmHg; deep coma; isocoria of two sides, with 0.2 cm diameter; no light reflex; barrel chest dry rales could be heard in two lungs.

Neural System examination: Absence of left eyelashes reflex and corneal reflex; muscular tension was not normal; especially both lower extremities. His muscle strength or tendon reflex could not be examined due to coma.

Diagnosis:

1. Pontine Hemorrhage, hematoma size was 10.88 ml.

2. Chronic Bronchitis accompanying Lung Infection

3. Chronic Obstructive Emphysema

Treatment and efficacy:

On admission, the patient was breathing normally and had normal blood pressure. He was treated for temperature, respiration, blood pressure, and ECG monitoring, oxygen supply, urethral catheterization and mannitol by vein injection for dehydration, at 125 ml per 4 hours. The patient was supplied nutrition and PHY828 (30 ml, per 6 hours) by stomach catheterization. He received PHY828 treatment for about six months.

Twenty-eight hours after admission, the patient's breath became slower and superficial. The worst was 4 times/min (respiratory failure). Further, there were bloody drainage liquid suggesting upper gastrointestinal hemorrhage. Losec (H⁺-blockervia in vein, 40 mg) and mechanical ventilation were administered simultaneously.

Mechanical ventilation was terminated 8 days later because his breathing became normal. But the breathing became unstable 75 hours after the first mechanical ventilation was removed. Thus, the mechanical ventilation was resumed for 60 hours.

The volume of manniol was reduced daily by monitoring the intracranial pressure (ICP), which was decreased to a normal level 13 days post admission. In total, mannitol was used for 13 days.

Thirty-four days after admission, the patient's consciousness changed for the better from coma to alert. He could turn his head or open his eyes in response to call. He also began to respond to light. He had sensitive light reflex and eyelash reflex. But he could not finish the action following the order directed by doctors. Anepia, and examination of muscle power could not be cooperated.

One hundred twenty days after admission, a second CT scan was performed. It showed that the hematoma and mass effect had disappeared (e.g. mass effect means midline drift, cerebral cisterna compressed, brain stem extruded to the left side, etc.).

One hundred twenty-seven days after admission, the patient was able to finish the action ordered by doctor, for instance, lifting his feet, extending his tongue etc. The muscle power of his left limbs was IV degree, and that of right limbs was I degree.

One hundred fifty-one days post admission the patient started to speak.

Six months after admission, the patient could stand up by himself and communicate verbally with his family members.

Ischemic Stroke Treated with PHY828 Botanical Drug

Two cases of ischemic stroke (Table 4-1˜4-3, Case #24, #25) were also treated with PHY828. As an example, Case #24, who previously had ischemic stroke in her left brain, suffered right brain ischemic stroke and was admitted. She required full time care upon admission. She was treated with PHY828. Ninety days following initial PHY828 treatment, there was significant recovery of her neural functions (NIHSS from 35 to 10; GOS from 3 to 2).

Pituitary Tumor Hemorrhage Treated with PHY828 Medicine

Besides those patients in Table 4-14˜3, a patient with pituitary tumor hemorrhage was also treated with PHY828. A 45 year old man was diagnosed with pituitary tumor found in February, 2000. The MRI showed that the tumor had ruptured and was bleeding. The tumor bleeding was completely absorbed after 30 days of PHY828 treatment, and his clinical problems were gone. The patient returned to work without any disability.

It should be understood that the foregoing discussion and examples merely present a detailed description of certain preferred embodiments. It therefore should be apparent to those of ordinary skill in the art that various modifications and equivalents can be made. Patent applications and references that are identified above are incorporated by reference in their entirety.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and in general, the principles of the invention and including such departures from the present disclosure as come within known or customary practice within the art to which the invention pertains and as may be applied to the essential features hereinbefore set forth and as follows in the scope of the appended claims.

1. A composition consisting essentially of materials from plant species of the following genera of herbs: one species of Ligusticum, two different species of Panax, and one species of Rheum.

2. A composition of claim 1, wherein the plant species are Ligusticum chuangxiong, Panax ginseng, Panax notoginseng and Rheum palmatum.

3. A composition of claim 2, wherein the plant species of Panax ginseng is replaced with Panax quinquefolium L.

4. A composition of claim 2, wherein Ligusticum chuanxiong is replaced with Ligusticum chuanxiong Hort, or Ligusticum officinale.

5. A composition of claim 1, further comprising a carrier.

6. A composition of claim 1, wherein said materials of Ligusticum, Panax, Panax, and Rheum having a dried weight ratio ranging from about 1:1:1:1 to about 10:10:10:10.

7. A composition of claim 6, wherein said materials having a dried weight ratio of about 2:2:2:1, respectively.

8. A composition of claim 1, wherein said composition is in an ingestible form or an injectible form.

9. A composition of claim 8, wherein the ingestible form is selected from powder, liquid, capsule, and tablet.

10. A method of making the composition of claim 1 comprising,
a) mixing and boiling in water, plant materials from Ligusticum and Panax to obtain a solution;
b) boiling separately in water, plant materials from Rheum to obtain a solution;
c) cooling, filtering and sterilizing the solutions, and mixing the sterilized solutions together to obtain the composition.

11. A method of making the composition of claim 1 comprising,
a) mixing and boiling in water, plant materials from Ligusticum, and Panax to obtain a solution;
b) adding plant materials from Rheum to the solution in step a) at about the last 10-15 minutes of boiling;
c) cooling, filtering and sterilizing the solution to obtain the composition.

12. A method of making the composition of claim 1 comprising preparing an extract with water from two different plant species of Panax, from one plant species of Ligusticum, and from one plant species of Rheum to obtain the composition.

13. A method of making the composition of claim 1 comprising,
a) preparing an extract from two different plant species of Panax comprising boiling Panax in alcohol to obtain an extract and filtering and concentrating the extract;
b) preparing an extract from one plant species of Ligusticum comprising boiling ligusticum in water, collecting condensate, adding surfactant to the condensate to obtain an aqueous solution, and filtering and concentrating the aqueous solution to obtain an extract;
c) preparing an extract from one plant species of Rheum comprising boiling plant materials from Rheum in water to obtain an extract;
d) mixing the three extracts together and sterilizing the mixture to obtain the composition.

14. A method of making the composition of claim 1 comprising preparing an extract of two different plant species of Panax and one plant species of Ligusticum with organic solutions and an extract of one plant species of Rheum with water to obtain the composition.

15. A method of treating a cerebral vascular disorder by administering to a mammal in need thereof, a therapeutically effective amount of a composition of claim 1.

16. The method of claim 15, wherein said cerebral vascular disorder is selected from the group consisting of head injuries, stroke, intracerebral hemorrhage, intracranial infarction and vascular dementia.

17. The method of claim 16, wherein said stroke is ischemic or hemorrhagic stroke.

18. The method of claim 15, wherein the mammal is a human.

19. The method of claim 15, wherein said treatment further comprises monitoring the improvement of the mammal by obtaining indices based on quality of life.

20. The method of claim 15, wherein said treatment further comprises monitoring the improvement of the mammal by obtaining indices based on mortality rate.

21. A composition consisting of materials from plant species of the following genera of herbs: one species of Ligusticum, two different species of Panax and one species of Rheum.

22. The composition of claim 21, wherein the plant species are Ligusticum chuangxiong, Panax ginseng, Panax notoginseng and Rheum Palmatum.

23. A method of making a composition comprising mixing plant materials consisting essentially of one species of Ligusticum, two different species of Panax, and one species of Rheum.

24. The method of claim 23, wherein the plant materials comprise raw herbs.

25. The method of claim 24, wherein the raw herbs are in the form of dry powder.

26. The method of claim 23, wherein the plant materials comprise extracts.

27. The method of claim 26, wherein the extracts are in the form of dry powder.

28. The method of claim 26, wherein the extracts are in tincture form.

29. The method of claim 26, wherein the extracts are prepared by extraction with water.

30. The method of claim 26, wherein the extracts are prepared by extraction with alcohol.

31. The method of claim 23, wherein the plant materials comprise whole plant.

32. The method of claim 23, wherein the plant materials comprise root.

33. The method of claim 23, wherein the plant materials comprise leaves.

34. The method of claim 23, wherein the plant materials comprise a combination of whole plant, root, and leaves.

35. A method of stimulating growth of neurocytes comprising administering the composition of claim 1 to a mammal.

36. A method of stimulating growth of neural tissues comprising administering the composition of claim 1 to a mammal.

37. A method of regenerating injured neural tissues comprising administering the composition of claim 1 to a mammal.

38. A method of regenerating injured neurocytes in a mammal comprising administering the composition of claim 1 to a mammal.

39. A method of decreasing intracranial pressure in a mammal in need thereof comprising administering the composition of claim 1 to the mammal.

40. A method of alleviating brain edema in a mammal in need thereof comprising administering the composition of claim 1 to the mammal.

41. A method of stimulating growth in neurocytes comprising administering the composition of claim 1 to neurocytes.

42. A method of regenerating injured neurocytes comprising administering the composition of claim 1 to injured neurocytes
